(19) United States
(12) Patent Application Publication
(10) Pub. No.: US 2007/0249546 A1
(43) Pub. Date: Oct. 25, 2007

(54) OPHTHALMIC AND RELATED AQUEOUS SOLUTIONS CONTAINING ANTIFUNGAL AGENTS, USES THEREFOR AND METHODS FOR PREPARING THEM

(76) Inventor: Assad S. Sawaya, Baiting Hollow, NY (US)

Correspondence Address: DARBY & DARBY P.C., P.O. BOX 770, Church Street Station, New York, NY, 10008-0770, US

(21) Appl. No.: 11/738,451

(22) Filed: Apr. 20, 2007

Related U.S. Application Data
(60) Provisional application No. 60/794,240, filed on Apr. 22, 2006.

(51) Int. Cl. A61K 31/7048; A61K 31/496; A61K 31/4196; A61K 31/4178

(52) U.S. Cl. 514/28; 514/254.07; 514/383; 514/397; 514/772.7

(57) ABSTRACT

The invention relates generally to concentrates and aqueous solutions for topical application comprising antifungal additives or agents as well as to preparation and use of such concentrates and solutions. More specifically, the invention relates to preparation and use of solutions that come in contact with the eye lids and/or eyes, such as but not limited to contact lens solutions, aqueous ophthalmic rinse solutions, and aqueous surgical scrubs for ophthalmic use.

### REFERENCE TO PROVISIONAL APPLICATION

This application claims the benefit of U.S. Provisional Application No. 60/794,240, filed on Apr. 22, 2006.

### FIELD OF THE INVENTION

The invention relates generally to concentrates and aqueous solutions for topical application comprising antifungal additives or agents as well as to preparation and use of such concentrates and solutions. More specifically, the invention relates to preparation and use of solutions that come in contact with the eye lids and/or eyes, such as but not limited to contact lens solutions, aqueous ophthalmic rinse solutions, and aqueous surgical scrubs for ophthalmic use.

### BACKGROUND OF THE INVENTION

In conventional antifungal preparations for ophthalmic or periophthalmic use, the antifungal agent is suspended in ointments, creams and non-aqueous solutions of oil-based delivery systems. Conventional antifungal agents precipitate when introduced into an aqueous solution, which diminishes or extinguishes their antifungal properties, rendering such agents impractical for use in aqueous solutions such as, but not limited to, multipurpose lens solutions.

The prior art lacks methods of preparing ophthalmic preparations that are homogenous aqueous solutions comprising antifungal agents for use in and around the eye. Instead, the prior art provides ophthalmic preparations comprising antifungal agents in the form of suspensions. For example, U.S. Pat. No. 7,056,893 (hereafter the '893 patent) discloses using a polymeric suspending agent like polyethylene glycol to prepare an aqueous polymeric suspension, However, a skilled worker will appreciate that a polymeric suspension is not the equivalent of a clear, homogenous aqueous solution, especially for use with the eye. Published U.S. patent application No. US2004/0198829 discloses the addition of prostanoids to a suspension comprising a therapeutic agent like an antifungal such as voriconazole to effectively increase transport and/or penetration of the therapeutic agent in the eye. However, neither the '893 patent nor US2004/0198829 provides a homogenous ophthalmic or periophthalmic aqueous solution comprising an effective amount of an antifungal agent.

Published U.S. patent application No. US2004/0266702, another example in the prior art, describes an ophthalmic composition that uses a polymeric suspension agent such as a carboxy-containing polymer such as polymers of acrylic acid to deliver to the eye an azalide antibiotic alone or in combination with another agent like an antifungal agent such as miconazole nitrate. US2004/0266702 discloses that the polymeric suspension agent may be polyethylene glycol and may additionally comprise a surfactant. The preparations disclosed in US2004/0266702 are aqueous polymeric suspensions. US2004/0266702 is silent as to a suitable method comprising steps for obtaining an aqueous solution comprising an azalide antibiotic and/or an antifungal agent without precipitation of the azalide antibiotic or antifungal agent. Thus, prior to the current invention, antifungal agents could not be used and were not contemplated for use in aqueous solution preparations for ocular use because they were inadequately soluble or even dispersible.

Conventional ophthalmic solutions and pre-surgical eye lid scrubs suffer from similar deficiencies vis a vis protection against fungal contamination. Such solutions and scrubs are vulnerable to similar fungal contamination, which leaves subjects using the solution and/or scrubs vulnerable to fungal infection. As a result of such contamination, subjects using conventional multipurpose lens solutions and/or conventional ophthalmic aqueous solutions and pre-surgical eye lid scrubs may develop conditions such as Fusarium keratitis eye infections and/or Fusarium conjunctivitis. Some infections may result to damage to the cornea. In some cases, corneal transplants are required, and on some occasions the subjects suffer loss of sight.

The use point, the eye, precludes organic solvents, such as alcohols, because they are irritant or toxic. While conventional multipurpose lens solutions contain preservative systems, recent events have demonstrated that such solutions are still vulnerable to contamination by fungal species such as Fusarium solani, which leaves the subject using conventional solutions vulnerable to fungal infection.¹
¹See FDA News, Apr. 10, 2006 reporting the presence of Fusarium in multipurpose contact lends solutions manufactured by Bausch & Lomb resulting in 109 cases of Fusarium keratitis. See FDA Update regarding Contact Lenses and Eye Infections dated Apr. 13, 2006 and FDA Statement dated Apr. 14, 2006 regarding voluntary recall of Bausch & Lomb Contact Lens Solution.

Heretofore no one has come forward with a method for providing an effective amount of an antifungal agent in an aqueous ophthalmic or periophthalmic solution, wherein the solution is physiologically acceptable for ophthalmic or periophthalmic use. Nor has a preparation containing an antifungal agent in solution in a suitable physiologically acceptable vehicle been devised such that it can be mixed with an aqueous ophthalmic or periophthalmic solution and impart to such a solution antifungal properties without precipitating the antifungal agent. Thus, a need for a safe antifungal preparation formulated for delivery in the form of a solution that may be used in aqueous solutions, such as multipurpose lens solutions and ophthalmic, topical and surgical solutions, is readily apparent. The antifungal preparations in the form of an aqueous solution presented herein will be effective and safe to reduce the risk of various fungal infections inherent in the currently available multipurpose lens solutions, and ophthalmic, topical and pre-surgical solutions, which lack the benefit of the technology presented herein. The antifungal preparations in the form of an aqueous solution presented herein may also increase the shelf life of conventional ophthalmic solutions and pre-surgical eye lid scrubs.

### BRIEF SUMMARY OF THE INVENTION

In accordance with this invention, there is provided a method for preparing a concentrate solution containing an antifungal agent in a form suitable for subsequent introduction in an aqueous medium suitable for ophthalmic or periophthalmic use, the method comprising the steps of: a) dissolving an antifungal agent in a physiologically acceptable non-aqueous vehicle to form a non-aqueous solution of said antifungal agent said vehicle being miscible with aqueous solutions; b) adding a physiologically acceptable surfactant to said non-aqueous solution in an amount sufficient to stabilize said antifungal agent upon its subsequent introduction in said aqueous medium; and c) mixing the non-aqueous solution and surfactant mixture until a clear concentrate solution is obtained.

The present invention also provides a method for inhibiting fungal growth in an aqueous solution suitable for ophthalmic or periophthalmic use comprising the step of dissolving in said aqueous solution a concentrate solution containing an antifungal agent dissolved in a physiologically acceptable non-aqueous vehicle, said vehicle being miscible with said aqueous solution and one or more physiologically acceptable surfactants, said concentrate solution being added to said aqueous solution in an amount sufficient to provide a fungicidally or fungistatically effective amount of said antifungal agent in said aqueous solution.

The invention provides a concentrate solution containing an antifungal agent in a form suitable for subsequent introduction in an aqueous medium suitable for ophthalmic or periophthalmic use, said concentrate solution made by: a) dissolving an antifungal agent in a physiologically acceptable non-aqueous vehicle to form a non-aqueous solution of said antifungal agent said vehicle being miscible with aqueous solutions; b) adding a physiologically acceptable surfactant to said non-aqueous solution in an amount sufficient to stabilize said antifungal agent upon its subsequent introduction in said aqueous medium; and c) mixing the non-aqueous solution and surfactant mixture until a clear concentrate solution is obtained.

The invention also provides an aqueous solution for ophthalmic or periophthalmic use comprising an antifungal agent in an amount effective to inhibit fungal growth in said aqueous solution, said solution made by dissolving in a first aqueous solution containing no antifungal agent a second concentrate solution containing (a) an antifungal agent dissolved in a physiologically acceptable non-aqueous vehicle, said vehicle being miscible with said first aqueous solution containing no antifungal agent; and (b) one or more physiologically acceptable surfactants; said concentrate having been added to the first aqueous solution containing no antifungal agent in an amount sufficient to provide a fungicidally or fungistatically effective amount of said antifungal agent in the combined solution.

In some embodiments, the invention provides a concentrate solution for ophthalmic or periophthalmic use containing from about 0.005% (w/w) to about 20.0 % (w/w) antifungal agent, from about 20% (w/w) to about 80% (w/w) non-aqueous vehicle, and from about 20% (w/w) to about 70.0% (w/w) surfactant.

In other embodiments, the invention provides an aqueous solution for ophthalmic or periophthalmic use comprising from about 0.001% (w/w) to about 2.0% (w/w) of an antifungal agent such as miconazole nitrate or tolnaftate, from about 0.5% (w/w) to about 2.0% (w/w) of a vehicle such as polyethylene glycol 400, from about 0.3% (w/w) to about 10.0% (w/w) of a surfactant such as polysorbate 80 and, optionally, from about 0.3% (w/w) to about 10.0% (w/w) of another surfactant such as octoxynol 40. In other embodiments, the aqueous solutions of the invention may additionally comprise additional agents such as polyquaternium-1, polyhexamethylene biguanide.

The invention also provides a method for avoiding fungal infection in a subject, said infection arising from contact of the eye of said subject with an aqueous solution during ophthalmic or periophthalmic use of said aqueous solution, the method comprising: providing as the aqueous solution used ophthalmically or periophthalmically an aqueous solution suitable for ophthalmic or periophthalmic use to which has been added a concentrate comprising (a) an antifungal agent dissolved in a non-aqueous vehicle, said vehicle being miscible with an aqueous solution suitable for ophthalmic or periophthalmic use containing no antifungal agent, and (b) one or more physiologically acceptable surfactants, said concentrate having been added in an amount sufficient to provide a fungicidally or fungistatically effective amount of said antifungal agent in said provided aqueous solution.

### BRIEF DESCRIPTION OF DRAWINGS

FIGS. 1-7 are photographs of plates showing the growth of Fusarium solani in the absence (FIG. 1) or presence (FIGS. 2-7) of aqueous solutions comprising antifungally effective amounts of tolnaftate or miconazole nitrate.

FIG. 8 is a graph of data collected from a disinfecting efficacy test using antifungal triplex hydrated solutions (ATHE-1 (FIG. 8A), ATHE-2 (FIG. 8B) and ATHE-3 (FIG. 8C)) to reduce the growth of Fusarium solani. FIG. 9 is a graph of the growth reduction data from the disinfecting efficacy test shown as log reductions.

### DETAILED DESCRIPTION OF THE INVENTION

As used herein and in the appended claims, the singular forms a “an”, and “the” include plural referents unless the context clearly dictates otherwise. Thus, for example, reference to “an antifungal agent” includes a plurality of such agents known to those skilled in the art, and so forth.

Unless defined otherwise, all technical and scientific terms used herein have the same meaning, as commonly understood to one of ordinary skill in the art to which this invention belongs.

The term “antifungal agent” refers to a substance capable of imparting fungistatic and/or fungicidal properties.

The term “solution” refers to one or more solutes that are completely dissolved or dispersed in a solvent yielding a homogenous mixture.

In some embodiments, the subject is a human. In some embodiments, the subject is a veterinary subject or an experimental animal, e.g., a rodent.

I. Aqueous Solutions Comprising Antifungal Agents and Methods of Preparing the Same

Aqueous ophthalmic solutions containing antifungal agents avoid the risk of various fungal infections inherent in currently available solutions, such as, but not limited to, multipurpose lens solutions, and ophthalmic, topical and pre-surgical solutions, because these conventional solutions do not contain antifungal agents. The present invention provides aqueous solutions comprising effective amounts of antifungal agents that remain in solution and impart antifungal properties to the solutions while maintaining these solutions as physiologically acceptable (e.g., nonirritant and nontoxic). The aqueous solutions are homogenous and clear. The aqueous solutions comprising effecting amounts of antifungal agents are not suspensions. The present invention provides aqueous solutions for ophthalmic or periophthalmic use comprising physiologically acceptable, yet effective, amounts of antifungal agents.

While not intending to be bound by any theory, it is believed that the described antifungal concentrates and aqueous solutions comprising antifungal agents maintain the antifungal agents in solution with low to no precipitation by use of a non-aqueous, but water soluble, physiologically acceptable solvent for the antifungal agent (e.g., polyethylene glycol) and one or more stabilizing agents like a physiologically acceptable surface active agent (e.g., a surfactant). It also is believed that the antifungal properties are maintained in the aqueous solution comprising an effective amount of antifungal agents because the antifungal agents substantially remain in solution.

A. Antifungal Concentrate

The present invention employs an antifungal concentrate comprising an antifungal agent, a physiologically acceptable non-aqueous vehicle in which the agent is soluble, and a physiologically acceptable surfactant. In one embodiment, the invention provides a method for preparing a concentrate comprising the steps of: 1) dissolving one or more antifungal agents in a physiologically acceptable non-aqueous vehicle; 2) adding one or more physiologically acceptable surfactants; and 3) mixing (e.g., by stirring) until a clear solution is obtained. In one embodiment, the invention provides a method of preparing a concentrate comprising the steps of: 1) applying heat (within the range of 50° C.-85° C. that is sufficient to dissolve an antifungal agent into a physiologically acceptable non-aqueous vehicle without destroying the antifungal properties of the antifungal agent; 2) adding a physiologically acceptable surfactant (within the temperature range of 30° C.-40° C. with continual mixing); and 3) mixing until a clear surfactant and antifungal containing solution is obtained. The mixing and dissolving steps can be combined. In a preferred embodiment of the method of preparing the antifungal concentrate of the invention, the antifungal agent is dissolved into the physiologically acceptable non-aqueous vehicle without destroying the antifungal properties of the antifungal agent while applying heat within the range of 75° C.-80° C.

In another embodiment, the invention provides a method of preparing an aqueous solution suitable for ophthalmic or periophthalmic use that comprises an effective amount of an antifungal agent dissolved therein. An effective amount of the antifungal agent remains in solution imparting antifungal properties to the solution throughout the useful life of the solution. In one embodiment, an antifungal concentrate of the invention is added to a topical aqueous solution to form an aqueous solution having antifungal properties. In another embodiment, the antifungal concentrate is added to a topical aqueous solution. Preferably, an effective amount of the antifungal agent remains in solution for the useful life of the solution. The useful shelf life of the antifungal concentrate or an aqueous solution comprising the antifungal concentrate is storage-stable at room temperature of at least about 24 months or greater. In another embodiment, the invention provides a method of imparting antifungal properties to a topical aqueous solution of ophthalmic or periophthalmic use. In another embodiment, the aqueous solution comprising an antifungal agent is functionally stable for at least the minimum reasonable shelf life of such products.

Antifungal agents which can be used herein are physiologically acceptable at the effective amounts employed and, particularly suitable for ophthalmic or periophthalmic uses. Antifungal agents for use in the antifungal concentrates and/or topical aqueous solutions comprising effective amounts of antifungal agents include, but are not limited to amorolfine, amphotericin B, anidulafungin, butoconazole, butenafine, caspofungin, ciclopirox olamine, clotrimazole, econazole, fluconazole, flucytosine, griseofulvin, haloprogin, itraconazole, ketoconazole, micafungin, miconazole (including miconazole nitrate), naftifine, nikkomycin Z, nystatin (topical and liposomal), oxiconazole, posaconazole, pimaricin, ravuconazole, sulconazole, terbinafine, terconazole, tioconazole, tolnaftate, undecylenate, voriconazole, or any other antifungal agent or a salt thereof known to those of skill in the art may be used. The antifungal agents used in the invention include free acid, free base, salts and esters. In one embodiment, the antifungal agent is tolnaftate. In one embodiment, the antifungal agent is miconazole nitrate.

In all embodiments of the antifungal concentrate, the antifungal agents may be used in effective concentrations generally ranging from about 0.005% (w/w) to about 6.0% (w/w). Higher concentrations are permitted subject to the amounts of physiological acceptability, but are not necessary. In one embodiment of the antifungal concentrate, an antifungal agent may be used in concentrations ranging from about 0.01% (w/w) to about 4.0% (w/w). In one embodiment of the antifungal concentrate, the antifungal agent may be used in a concentration ranging from about 0.06 % (w/w) to about 2.0% (w/w). In one embodiment of the antifungal concentrate, miconazole nitrate may be used at a concentration of 0.1% (w/w) or 2.0% (w/w). In one embodiment, tolnaftate may be used at a concentration of 0. 1% (w/w) or 2.0% (w/w).

In all embodiments of the antifungal topical aqueous solution, antifungal agents may be used in effective concentrations generally ranging from about 0.001% (w/w) to about 2.0% (w/w). Higher concentrations are permitted subject to the amounts of physiological acceptability, but are not necessary. In one embodiment of the antifungal topical aqueous solution, an antifungal agent may be used in concentrations ranging from about 0.005% (w/w) to about 0.05% (w/w). In one embodiment of the antifungal topical aqueous solution, the antifungal agent may be used in a concentration ranging from about 0.01 % (w/w) to about 0. 1% (w/w). In one embodiment of the antifungal topical aqueous solution, miconazole nitrate may be used at a concentration of 0.01% (w/w) or 0.02% (w/w). In one embodiment of the antifungal topical aqueous solution, tolnaftate may be used at a concentration of 0.01% (w/w) or 0.02% (w/w). The solubility of each antifungal agent used in the current invention in a physiologically acceptable non-aqueous vehicle can be determined using methods well known in the art. Additionally, for each antifungal agent used in the current invention specific determinations can be conducted to determine the concentration of surfactant sufficient to aid in dissolution of the antifungal agent.

In all embodiments, the antifungal agent used is nontoxic and nonirritant (i.e. does not cause ocular irritation of the lens or conjunctiva of the eye) when administered to a subject. In all embodiments, the antifungal agent is used at a concentration that is safe for administration to a subject. In all embodiments, the concentration of the antifungal agent used by a method of the invention is nontoxic for the duration of administration to a subject.

Physiologically acceptable non-aqueous vehicles that may be used to dissolve an antifungal(s) include, without limitation: polyols, polyhydric alcohol polymers, polyhydric alcohol ethers, polyhydric alcohol esters. The non-aqueous vehicles are soluble in water. In some embodiments, the polyhydric alcohol ether is polyethylene glycol. In some embodiments, the polyethylene glycol has an average molecular weight generally in the range from about 4 to about 160,000. In other embodiments, the polyethylene glycol has an average molecular weight generally in the range from about 8 to about 1000. In other embodiments, the polyethylene glycol has an average molecular weight generally in the range from about 100 to about 600. In other embodiments, the polyethylene glycol has an average molecular weight generally in the range from about 60 to about 100.

Vehicles or solvents which may be used herein include, but are not limited to, polyethylene glycol 4, polyethylene glycol 6, polyethylene glycol 7, polyethylene glycol 8, polyethylene glycol 9, polyethylene glycol 10, polyethylene glycol 12, polyethylene glycol 14, polyethylene glycol 16, polyethylene glycol 18, polyethylene glycol 20, polyethylene glycol 32, polyethylene glycol 40, polyethylene glycol 45, polyethylene glycol 55, polyethylene glycol 60, polyethylene glycol 75, polyethylene glycol 90, polyethylene glycol 100, polyethylene glycol 135, polyethylene glycol 150, polyethylene glycol 180, polyethylene glycol 200, polyethylene glycol 220, polyethylene glycol 240, polyethylene glycol 350, polyethylene glycol 400, polyethylene glycol 500, polyethylene glycol 600, polyethylene glycol 800, polyethylene glycol 2000, polyethylene glycol 5000, polyethylene glycol 7000, polyethylene glycol 9000, polyethylene glycol 14000, polyethylene glycol 20000, polyethylene glycol 23000, polyethylene glycol 45000, polyethylene glycol 90000, polyethylene glycol 115M, polyethylene glycol 160M, propylene glycol, propylene glycol alginate, propylene glycol behenate, propylene glycol butyl ether, propylene glycol capreth-4, propylene glycol caprylate, propylene glycol ceteth-3 acetate, propylene glycol ceteth-3 propionate, propylene glycol citrate, propylene glycol coate, propylene glycol dicaprate, propylene glycol dicaproate, propylene glycol dicaprylate, propylene glycol dicocoate, propylene glycol diethyl hexanoate, propylene glycol diisononanoate, propylene glycol diisostearate, propylene glycol dilaurate, propylene glycol dioleate, propylene glycol dipelargonate, propylene glycol distearate, propylene glycol hydroxystearate, propylene glycol isoceteth-3 acetate, propylene glycol isostearate, propylene glycol laurate, propylene glycol laureth-6, propylene glycol linolenate, propylene glycol myristate, propylene glycol myristyl ether, propylene glycol myristyl ether acetate, propylene glycol oleate, propylene glycol oleate SE, propylene glycol oleth-5, propylene glycol propyl ether, propylene glycol stearate, propylene glycol stearate SE, or any other vehicles or solvents suitable in ophthalmic solutions as described herein that are known to those of skill in the art may be used. In one embodiment, the vehicle or solvent is polyethylene glycol 400.

To aid in dissolution of the concentrates including the antifungal agent(s) dissolved therein in an aqueous solution, the present invention employs surface-active agents (i.e. surfactants). In all embodiments, the surfactant(s) used herein is physiologically acceptable and in preparation of a topical aqueous solution comprising an antifungal agent. In some embodiments, the physiologically acceptable surfactants are suitable for ophthalmic and periophthalmic uses. In all embodiments, the surfactant(s) used in the present invention include, but are not limited to, nonionic and cationic surfactants. Anionic surfactants should be avoided.

Nonionic or neutral surfactants that may be used herein include polysorbates and polyethoxylates. In some embodiments, nonionic surfactants to be used herein include, but are not limited to, polysorbate 20, polysorbate 40, polysorbate 60, polysorbate 61, polysorbate 65, polysorbate 81, polysorbate 85, polysorbate 80, polysorbate 80 acetate, octoxynol 1, octoxynol 3, octoxynol 5, octoxynol 6, octoxynol 7, octoxynol 8, octoxynol 9, octoxynol 10, octoxynol 11, octoxynol 12, octoxynol 13, octoxynol 16, octoxynol 25, octoxynol 30, octoxynol 33, octoxynol 40, octoxynol 70, octoxynol-9 carboxylic acid, octoxynol-20 carboxylic acid, or any other surfactant suitable for ophthalmic and/or dermatologic purposes known to those of skill in the art may be used.

Suitable surfactants to be used herein include cationic surfactants such as quaternary ammonium compounds. In another embodiment, cationic surfactants used herein are, but are not limited to, benzalkonium chloride and benzalkonium saccharinate, or any other surfactant suitable for ophthalmic or periopthalmic purposes known to those of skill in the art may be used.

In some embodiments of the antifungal concentrate, surfactants may be used in concentrations ranging from about 20% (w/w) to about 70% (w/w). In some embodiments of the antifungal topical aqueous solution, surfactants may be used in concentrations ranging from about 0.3% (w/w) to about 10.0% (w/w). In a preferred embodiment of the antifungal topical aqueous solution, surfactants may be used in concentrations ranging from about 0.5% (w/w) to about 2% (w/w). In another preferred embodiment of the antifungal topical aqueous solution, surfactants may be used at a concentration of about 1.0% (w/w). In all embodiments, the surfactant used is nontoxic and nonirritant when administered to a subject. In all embodiments, the surfactant is used at a concentration that is safe for administration to a subject. In one embodiment, the surfactant is a combination of polysorbate 80 and octoxynol 40. In another embodiment, the surfactant is polysorbate 80. In another embodiment, the polysorbate 80 in the antifungal concentrate is used in a concentration of 48% (w/w). In another embodiment, the polysorbate 80 in the antifungal topical aqueous solution is used in a concentration of 1.0% (w/w). In another embodiment, the surfactant is octoxynol 40. In another embodiment, octoxynol 40 in the antifungal concentrate is used at a concentration of 50% (w/w). In another embodiment, the octoxynol 40 in the antifungal topical aqueous solution is used in a concentration of 2.0% (w/w). In one embodiment, the combination of polysorbate 80 and octoxynol 40 in the antifungal topical aqueous solution is used in a concentration of 1.0% (w/w).

B. Aqueous Solutions for Combination with the Antifungal Concentrates

One aspect of the present invention is to add the antifungal concentrate described herein to a first aqueous solution containing no antifungal agent to yield an aqueous solution imparting antifungal properties to said first aqueous solution without precipitating the antifungal agent. Another aspect of the present invention provides an aqueous solution for ophthalmic or periophthalmic use comprising an antifungal agent in an amount effective to inhibit fungal growth in the aqueous solution that is made by dissolving in a first aqueous solution containing no antifungal agent a second antifungal concentrate solution described herein. Aqueous solutions provided by the present invention for opthalmic or periophthalmic use comprising an antifungal concentrate described herein are homogenous. First aqueous solutions that may be employed to add to the antifungal concentrate of the invention include, but are not limited to, aqueous solutions which come into contact with the eye lids and/or eyes, such as multipurpose lens solutions, ophthalmic rinse solutions, surgical scrubs for eye use, eye drops, eye wash solutions, contact lens solutions, topical over the counter ocular and periocular solutions (i.e., artificial tears), ocular and periocular cleaning solutions, eye irrigating solutions, and/or antibacterial solutions for surgical scrubs or topical application. In other words, the first aqueous solution may contain one or more physiologically acceptable active ingredients such as the foregoing and/or excipients.

In some embodiments, an antifungal concentrate of the invention may be added to a commercially available contact lens solution or a multipurpose lens solution to impart antifungal properties without precipitation of the antifungal agent. In some embodiments, an antifungal concentrate of the invention may be added to an aqueous solution prepared for use as a contact lens or multipurpose lens solution that is not commercially available to impart antifungal properties without precipitation of the antifungal.

In some embodiments, an antifungal concentrate of the invention is added to an ocular or periocular cleaning solution to impart antifungal properties without precipitation of the antifungal agent. In some embodiments, the cleaning solution comprises cleaning agents to effectively clean a lens of film deposits and surface debris. Such cleaning agents that may be used include, without limitation, poloxamers and tetronic surfactants comprising poly(oxyethylene) hydrophilic units. In some embodiments, a topical aqueous solution that has been added to an antifungal concentrate of the invention additionally comprises one or more of the following cleaning agents in concentrations ranging from about 0.0001% (w/w) to 1.0% (w/w): Pluronic® F-127 (poloxamine), Tetronic® 904, Tetronic® 304, or other suitable cleaning agents. In all embodiments, the cleaning agents are nontoxic, and do not distort the vision of the subject using the antifungal concentrate or aqueous solution comprising the same.

In other embodiments, an antifungal concentrate of the invention may be added to tonicity agents and buffers that are found in conventional ophthalmic and periophthalmic solutions to impart antifungal properties without precipitation of the antifungal. In this regard, the components of said tonicity agents and buffers are nontoxic, and do not distort the vision of the subject using the antifungal concentrate. Suitable tonicifiers that may be added to the antifungal concentrates and/or aqueous solutions comprising antifungal agents include, without limitation, dextrose, potassium chloride and/or sodium chloride. Suitable buffers that may be added to the antifungal concentrate and/or aqueous solution comprising antifungal agents include, without limitation, boric acid, sodium borate, sodium or potassium citrate, sodium bicarbonate, sodium phosphate, and potassium phosphate. The buffers may be used in concentrates ranging from about 0.3% (w/w) to about 3.5% (w/w).

Additionally, to also guard against bacterial infection, antibacterial agents found in conventional ophthalmic solutions such as multipurpose lens solutions may be added to the antifungal concentrates and/or aqueous solutions comprising antifungal agents. In this regard, the antibacterial agents are nontoxic, and do not distort the vision of the subject using the antifungal concentrate or aqueous solution comprising the same. Antibacterial agents for use in the antifungal concentrates and/or aqueous solutions comprising antifungal agents include, but are not limited to, polyaminopropyl biguanide, alexidine hydrochloride, polyquaternium, polyquaternium 42, myristamidopropyl dimethylamine, or other suitable agents known to a skilled worker. In some embodiments, polyaminopropyl biguanide may be used in concentrations ranging from 0.0001% (w/w) to 0.002% (w/w) in the antifungal concentrate and/or aqueous solution comprising antifungal agents. In other embodiments, alexidine hydrochloride may be used in concentrations ranging from about 0.0002% (w/w) to about 0.006% (w/w) in the antifungal concentrate and/or aqueous solution comprising antifungal agents.

In some embodiments, the aqueous solutions comprising antifungal agents also comprise a comfort or moisturizing agent to provide hydration and lubrication of the lens. Such agents include, but are not limited to, polyquaternium 10, poloxamer, propylene glycol, hydroxypropylmethylcellulose (HPMC), or other suitable agents known to a skilled worker. In all embodiments, the cleaning agents are nontoxic, and do not distort the vision of the subject using the antifungal concentrate or aqueous solution comprising the same. In some embodiments, a comfort or moisturizing agent may be used in concentrations ranging from 0.01% (w/w) to 2.0% (w/w) in the antifungal concentrate and/or aqueous solution comprising antifungal agents. In a preferred embodiment, a comfort or moisturizing agent may be used in concentrations ranging from 0.1% (w/w) to 0.8% (w/w) in the antifungal concentrate and/or aqueous solution comprising antifungal agents.

Since, in some embodiments, the aqueous solutions comprising antifungal agents are intended to be administered topically to the eye lids and/or eye, it is preferred that they be free of pathogenic organisms and/or sterile. A benefit of a sterile solution is that it reduces the possibility of introducing contaminants into a subject when the antifungal concentrates and/or aqueous solutions comprising antifungal agents of the present invention are administered topically, for example, to the eye lids and/or eye. Sterility or adequate antimicrobial preservation may be provided as part of the present antifungal preparations. In some embodiments, the antifungal concentrates and/or aqueous solutions comprising antifungal agents of the present invention are produced under sterile conditions.

In lieu of or additional to sterilization, the aqueous solutions comprising antifungal agents may contain a physiologically acceptable preservative to minimize the possibility of microbial contamination. A physiologically acceptable preservative may be used in the present antifungal preparations to increase the stability of the antifungal preparations. Preservatives suitable for use herein include, but are not limited to, polyaminopropyl biguanide, polyhexamethylene biguanide (PHMB), polyquaternium-1, myristamidopropyl, and sorbic acid. In some embodiments, preservatives may be used in concentrations ranging from about 0.00005% (w/w) to about 0.00015% (w/w). In one embodiment, polyquatemium-1 may be used in concentrations ranging from about 0.001% (w/w) to about 0.002% (w/w). In another embodiment, myristamidopropyl (w/w) may be used at a concentration of about 0.0005% (w/w). In another embodiment, sorbic acid may be used in concentrations ranging from about 0.1% (w/w) to about 0.3% (w/w).

II. Uses for Aqueous Solutions Containing Antifungal Agents

Subjects who use the antifungal concentrates and/or aqueous solutions comprising antifungal agents produced by methods of the present invention may attain previously unavailable levels of protection against fungal organisms and species. As a result, subjects using such antifungal concentrates and/or aqueous solutions comprising antifungal agents produced by methods of the present invention may evade vulnerability to exposure to fungal organisms and may prevent or may avoid the development of fungal infections caused by fungal genera. The antifungal concentrates and/or aqueous solutions comprising antifungal agents made by methods of the invention are intended for administration to a subject to prevent or to avoid a fungal infection.

Fungal genera for which the antifungal concentrates and/or aqueous solutions comprising antifungal agents made by methods of the current invention may have activity against include, but are not limited to Fusarium, Penicillium, Aspergillus, Cephalosporium (Acremonium), Trichophyton, Microsporum, Epidermophyton, Scopulariopsis, and Candida. In some embodiments, a fungal genus against which the antifungal concentrates and/or aqueous solutions comprising antifungal agents may be used is Fusarium. In some embodiments, fungal species for which the present invention may have activity against include, but are not limited to: Fusarium solari; Fusarium solani, Fusarium avenaceum, Fusarium culmorum, Fusarium balbigenum, Fusarium caeruleum, Fusarium conglutinans, Fusarium lini, Fusarium oxysporum, Fusarium vasinfectum, Fusarium graminearum, Trichophyton crateriform, Trichophyton rubrum, Trichophyton mentagpophytes, Trichophyton interdigitalis, Trichophyton verrucosum, Trichophyton megnini, Trichophyton gallinae, Trichophyton sulphureum, Trichophyton schoenleini, Microsporum audonini, Microsporum canis, Microsporum gypsum, Epidermophytonfloccosum, Scopulariopsis brevicaulis, and combinations thereof. In some embodiments, a fungal species against which the antifungal concentrates and/or aqueous solutions comprising antifungal agents may be used is Fusarium solari.

Fungal infections that may be avoided with antifungal concentrates and/or aqueous solutions comprising antifungal agents of the invention are, but not limited to, eye conditions with a fungal-based etiology. In some embodiments, the Fusarium solani is responsible for the fungal-based etiology. In some embodiments, the antifungal concentrates and/or aqueous solutions comprising antifungal agents made by the methods of the invention may be used in a subject for the prevention of a fungal-based eye condition.

In other embodiments, fungal infections for which concentrates or aqueous solutions made by methods of the current invention may have activity against include, but are not limited to: Fusarium keratitis, Fusarium conjunctivitis, and endophthalmitis. In one embodiment, the fungal infection to be prevented or to be avoided is Fusarium keratitis. In one embodiment, the antifungal concentrates and/or aqueous solutions comprising antifungal agents made by the methods of the invention are administered for prevention of Fusarium keratitis. In another embodiment, the fungal infection to be prevented or to be avoided is Fusarium conjunctivitis. In one embodiment, the antifungal concentrates and/or aqueous solutions comprising antifungal agents made by the methods of the invention is administered for prevention of Fusarium conjunctivitis.

The antifungal aqueous solutions of the invention comprising antifungal agents may be administered topically or may be used, e.g., on contact lenses during storage. Most commonly, the solutions of the invention can be applied as eye drops, eye washes, eye lid scrubs and the like.

In order that this invention may be better understood, the following examples are set forth. These examples are for the purposes of illustration only and are not to be construed as limiting the scope of this invention in any manner.

### EXAMPLE I

An aqueous topical solution comprising antifungal agents was produced by a method of the invention as follows:

A. Antifungal Concentrate

An antifungal concentrate was prepared by dissolving 0.2 grams miconazole nitrate in 10.0 grams polyethylene glycol (PEG) 400 with continual mixing at a temperature of 75°-85° C. until the miconazole nitrate was completely solubilized. This non-aqueous solution was then cooled to 30° -40° C. Next, with continual stirring, 5.6 grams of Tween® 80 (polysorbate 80) was added followed by addition of 4.5 grams of octoxynol 40 to stabilize the concentrate.

B. Multipurpose Lens Solution

An aqueous multipurpose lens solution was prepared by adding 0.25 grams of disodium ethylenediaminetetraacetic acid (EDTA), 0.60 grams of sodium borate, 5.0 grams of boric acid, 9.0 grams of propylene glycol, 1.0 grams of Pluronic® F-127, 0.6 grams of polyquaternium 10 and 1.8 grams of sodium chloride to 859.35 grams of purified water.

C. Cleaning Solution

A cleaning solution was prepared by adding, while continually stirring, 0.5 grams of Tetronic® 904, 0.1 grams of Tetronic® 304 and 1.5 grams of polyaminopropyl biguanide to 100 grams of purified water. (Surfactants sold under the trademark Tetronic are tetrafunctional block copolymer nonionic surfactants terminating in primary hydroxyl groups.)

A final aqueous solution was prepared by mixing the antifungal concentrate A. with the multipurpose lens solution B to form a clear homogenous solution. The resulting aqueous solution was filter sterilized using a 0.22 micron filter. Another final aqueous solution also was prepared by mixing the antifungal concentrate A. with the cleaning solution C. to form a clear, homogenous solution. The resulting aqueous solution was filter sterilized using a 0.22 micron filter.

### EXAMPLE II

Aqueous topical solution 061360A (Lot 061360A) comprising 0.02% (w/w) miconazole nitrate was prepared as follows:

A 061360 antifungal concentrate was prepared by dissolving 0.2 grams miconazole nitrate in 16.0 grams PEG 400 with continual mixing at a temperature of 75°-80° C. until the miconazole nitrate was completely solubilized. The solution was cooled to 35° C.±5° C. Next, with continual stirring, 5.0 grams of Tween® 80 (polysorbate 80) was added followed by addition of 4.5 grams of octoxynol 40 to stabilize the concentrate.

A 061360A aqueous multipurpose lens solution containing 0.1 % PHMB was prepared by adding 0.25 gram of EDTA, 0.60 gram of sodium borate, 4.0 grams of boric acid, 9.0 grams of propylene glycol, 1.0 grams of Poloxamer 407, 0.5 gram of Tetronic® 904, 0.1 gram of Tetronic® 304, 0.6 gram of PHMB and 1.0 gram of sodium chloride one ingredient at a time to 956.35 grams of purified water with continual mixing and heating to 70° -80° C. After dissolution of all ingredients, the 061360A aqueous multipurpose lens solution containing 0.1% PHMB was then cooled to room temperature.

Lot 061360A was prepared by slowly adding the 061360 antifungal concentrate to the 061360A multipurpose lens solution containing 0.1% PHMB with continual mixing at 35° C. until a clear solution was obtained. Lot 061360A was cooled to room temperature. Lot 061360A is a clear, aqueous solution which contains 0.02% Miconazole Nitrate, 1.6% PEG 400, 0.5% Tween® 80 (polysorbate 80), 0.45% octoxynol 40, and 1 ppm PHMB.

### EXAMPLE III

Aqueous topical solution 061360B (Lot 061360B) comprising 0.02% (w/w) miconazole nitrate was prepared as follows:

A 061360B aqueous multipurpose lens solution containing 0.1% polyquaternium-1 was prepared by adding 0.25 gram of EDTA, 0.60 gram of sodium borate, 4.0 grams of boric acid, 9.0 grams of propylene glycol, 1.0 gram of Poloxamer 407, 0.5 gram of Tetronic®904, 0.1 gram of Tetronic®304, 12.0 grams of polyquatemium-1 and 1.0 gram of sodium chloride one ingredient at a time to 945.85 grams of purified water with continual mixing and heating to 70°-80° C. After dissolution of all ingredients, the 061360B aqueous multipurpose lens solution containing 0.1% polyquaternium-1 was then cooled to room temperature.

Lot 061360B was prepared by slowly adding the 061360 antifungal concentrate (described in Example II) to the 061360B multipurpose lens solution containing 0.1 % (w/w) polyquatemium-1 with continual mixing at 35° C. until a clear solution was obtained. Lot 061360B was cooled to room temperature. Lot 061360B is a clear, aqueous solution which contains 0.02% (w/w) Miconazole Nitrate, 1.6% (w/w) PEG 400, 0.5% (w/w) Tween® 80 (polysorbate 80), 0.45% (w/w) octoxynol 40, and 0.001% (w/w) polyquaternium-1.

### EXAMPLE IV

Aqueous topical solutions comprising antifungal agents prepared according to the methods of the present invention were used to test their fungicidal properties against Fusarium solani over a period of 24 hours.

Preparation of Fusarium Solani Culture Suspension

A Fusarium solani culture suspension was prepared as follows: From a working slant a culture of Fusarium solani was transferred to the surface area of a Roux bottle containing 200 mL of Sabourand Dextrose Agar. The Roux bottle and its contents were incubated at 20-25° C. for 3 to 7 days. Next, the surface area of the Roux bottle was rinsed with 50 mL of sterile saline solution, which subsequently was centrifuged at no more than 4000 ×g for a maximum of 15 minutes. To determine the culture population, serial dilutions were performed using Sabourand Dextrose Agar in a ratio of 1 to 10 at a minimum of 1×10⁻⁷ to 1 ×10⁻⁸ dilution. Each dilution was plated and incubated at 20-25° C. for 3 to 7 days. The number of Fusarium solani cfu per dilution was determined by counting the culture growth on each plate.

Treatment of Fusarium Solani with Aqueous Solutions Comprising 0.02% (w/w) Tolnaftate or 0.02% (w/w) Miconazole Nitrate

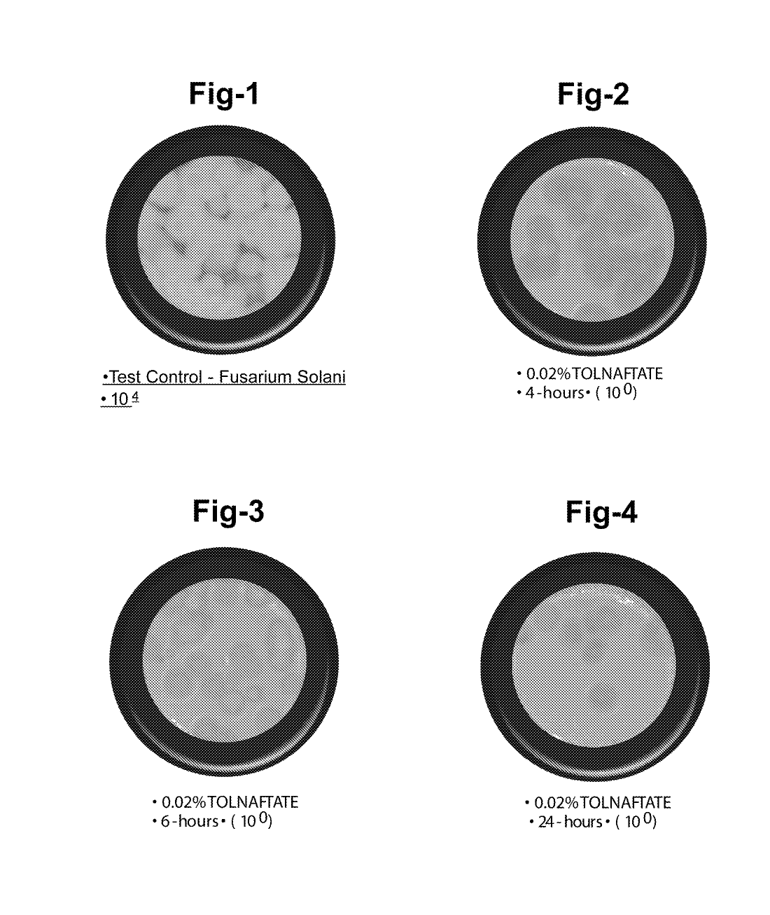

Inoculums of Fusarium solani culture solution that were used to conduct the experiments for which data is shown in FIGS. 1-7 was prepared by diluting 0.1 mL of a 1.66×10⁶ cfu/mL Fusarium solani culture suspension into 10 mL of aqueous solutions comprising 0.02% (w/w) tolnaftate or 0.02% (w/w) miconazole nitrate providing a final count of 1.66×10⁴ cfu/mL (typical range of 1×10⁴ to 1×10⁵ cfu/mL). The inoculated sample was stored at 20-25° C.

Viable counts of inoculated samples were determined at 1 hour, 2 hours, 3 hours, 4 hours, 6 hours, and 24 hours by removing 1.0 mL aliquots of the inoculated sample were used. To each 1.0 mL aliquot, 9 mL of phosphate buffered saline (PBS) containing 0.5 % Tween® 80 (polysorbate 80) was added to perform serial decimal dilutions. The viable count of Fusarium solani was determined by dilution into culture plates using recovery medium (Sabourand Dextrose Agar). The plates were incubated at 20-25° C. for 3 to 7 days. The number of Fusarium solani cfu was determined by counting the culture on the plates. Log reduction of Fusarium solani cfu was calculated for each specific time point.

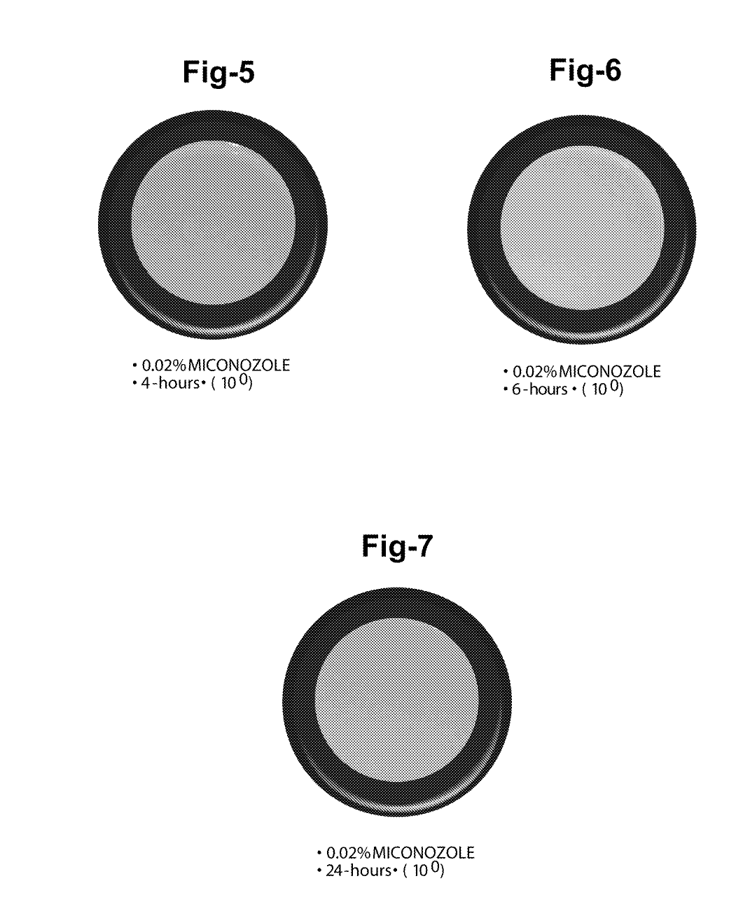

As discussed above, plates containing an initial inoculum comprising 1.66×10⁴ cfu Fusarium solani were monitored over a period of 24 hours in the absence (See FIG. 1) or presence (See FIGS. 2-7) of aqueous solutions comprising 0.02% (w/w) tolnaftate or 0.02% (w/w) miconazole nitrate, as prepared by methods of the invention. All plates that were treated with the aqueous solutions comprising 0.02% (w/w) tolnaftate or 0.02% (w/w) miconazole nitrate for a period of 4, 6 or 24 hours showed reduced growth of Fusarium solani. FIG. 2 is a photograph of a plate showing 30 cfu remaining after 4 hours treatment with 0.02% (w/w) tolnaftate, which represents a 3.39 log reduction in the growth of Fusarium solani. FIG. 3 is a photograph of a plate showing 30 cfu remaining after 6 hours treatment with 0.02% (w/w) tolnaftate, which shows a 3.46 log reduction in the growth of Fusarium solani. FIG. 4 is a photograph of a plate showing 10 cfu remaining 24 hours after treatment with 0.02% (w/w) tolnaftate, which represents a 3.94 log reduction in the growth of Fusarium solani. FIG. 5 is a photograph of a plate showing 0 (zero) cfu remaining 4 hours after treatment with 0.02% (w/w) miconazole nitrate, which represents a 4.91 log reduction in the growth of Fusarium solani. FIG. 6 is a photograph of a plate showing 0 (zero) cfu remaining 6 hours after treatment with 0.02% (w/w) miconazole nitrate, which represents a 4.91 log reduction in the growth of Fusarium solani. FIG. 7 is a photograph of a plate showing 0 (zero) cfu remaining 24 hours after treatment with 0.02% (w/w) miconazole nitrate, which represents a 4.91 log reduction in the growth of Fusarium solani.

### EXAMPLE V

Aqueous solutions comprising the following antifungal agents: 0.01% (w/w) tolnaftate (ATHE-1); 0.02% (w/w) tolnaftate (ATHE-2); or 0.02% (w/w) miconazole nitrate (ATHE-3) were prepared according to methods of the invention and were tested for their fungicidal properties against Fusarium solani in a disinfectant efficacy test.

The solution ATHE-1 was prepared by 1) dissolving 0.66% (w/w) tolnaftate in 33% (w/w) PEG 400 with continual mixing at a temperature of 75° C.-80° C. until the tolnaftate was completely solubilized; 2) cooling the non-aqueous solution to 35° C.±5° C.; 3) with continual stirring at a temperature of 35° C.±5° C., 36% (w/w) Tween® 80 was added; 4) then with continual stirring 35° C.±5° C., 30% (w/w) octoxynol 40 was added; 5) the homogenous non-aqueous solution was cooled to 25° C.±3° C. and 6) the resulting tolnaftate concentrate was added with continual mixing at a temperature of 25° C.±3° C. to purified water. ATHE-1 is a clear, aqueous solution for ophthalmic and periophthalmic use that contains 0.01% tolnaftate, 0.5% PEG 400, 0.55% Tween® 80, and 0.46% octoxynol 40.

The solution ATHE-2 was prepared by 1) dissolving 0.99% (w/w) tolnaftate in 49% (w/w) PEG 400 with continual mixing at a temperature of 75° C.-80° C. until the tolnaftate was completely solubilized; 2) cooling the non-aqueous solution to 35° C.±5° C.; 3) with continual stirring, 27% (w/w) Tween® 80 was added; 4) then with continual stirring at a temperature of 35° C.±5° C., 36% at 35° C.±5° C., 23% (w/w) oxtoxynol 40 was added; 5) the non-aqueous solution was cooled to 25° C. ±3° C.; and 6) the resulting tolnaftate concentrate was added with continual mixing at a temperature of 25° C.±3° C. to purified water. ATHE-2 is a clear, aqueous solution for ophthalmic and periophthalmic use that contains 0.02% tolnaftate, 1% PEG 400, 0.56% Tween® 80, and 0.46% octoxynol 40.

The solution ATHE-3 was prepared by 1) dissolving 0.99% (w/w) miconazole nitrate in 49% (w/w) PEG 400 with continual mixing at a temperature of 75° C.-80° C. until the miconazole nitrate was completely solubilized; 2) cooling the non-aqueous solution to 35° C.±5° C.; 3) with continual stirring at a temperature of 35° C.±5° C., 27 % (w/w) Tween® 80 was added; 4) then continual stirring at a temperature of 35° C.±5° C., 23% (w/w) oxtoxynol 40 was added; 5) the non-aqueous solution was cooled to 25° C.±3° C.; and 6) the resulting miconazole nitrate concentrate was added with continual mixing at a temperature of 25° C.±3° C. to purified water. ATHE-3 is a clear, aqueous solution for ophthalmic and periophthalmic use that contains 0.02% miconazole nitrate, 1% (w/w) PEG 400, 0.55% Tween® 80, and 0.46% octoxynol 40.

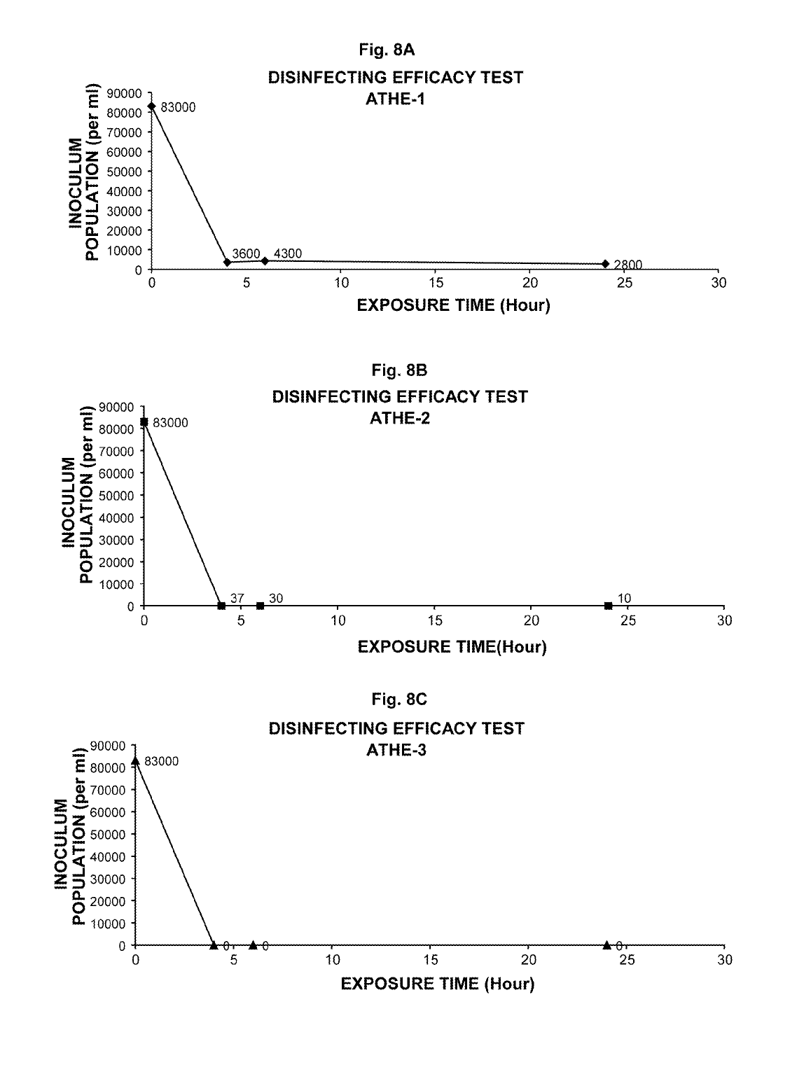

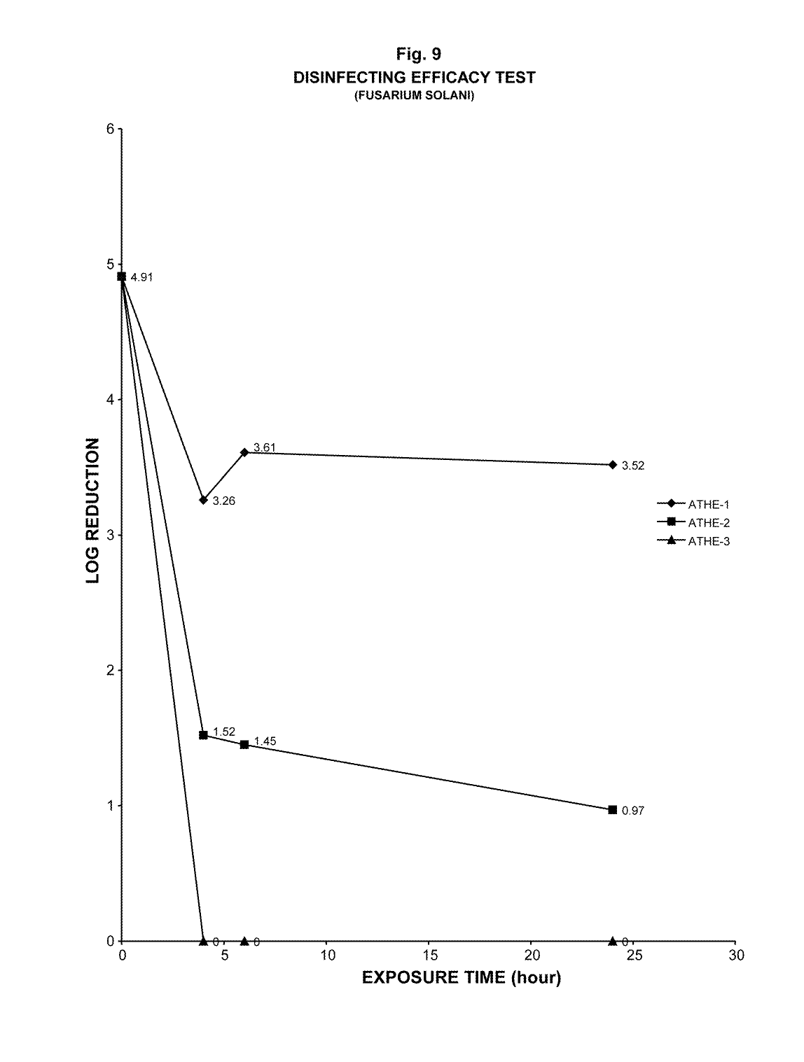

The aqueous solution comprising 0.02% (w/w) tolnaftate was added to an initial inoculum of 83,000 cfu per mL of Fusarium solani, and after four (4) hours of exposure time, the initial inoculum was reduced to 37 cfu (See Table 1 below and FIG. 8). This result demonstrates a log reduction of 3.39 in Fusarium solani growth after four (4) hours of exposure time (See Table 1 below and FIG. 9). The aqueous solution comprising 0.01% (w/w) tolnaftate was added to an initial inoculum of 83,000 cfu per mL of Fusarium solani, and after four (4) hours of exposure time, the initial inoculum was reduced to 3,600 cfu (See Table 1 below and FIG. 8). This result demonstrates a log reduction of 1.29 in Fusarium solani growth after four (4) hours of exposure time (See Table 1 below and FIG. 9). The aqueous solution comprising 0.02% (w/w) miconazole nitrate was added to an initial inoculum of 83,000 cfu per mL of Fusarium solani, and after four (4) hours of exposure time, the initial inoculum was reduced to zero (0) cfu (See Table 1 below and FIG. 8). This result demonstrates a log reduction of 4.91 in Fusarium solani growth after four (4) hours of exposure time (See Table 1 below and FIG. 9).

TABLE 1
Disinfectant efficacy test results for ATHE-1, ATHE-2, and ATHE-3 aqueous solutions

| | Exposure | F. solani Plate | | Log |
|---|---|---|---|---|
| Concentration/Ingredients | Time | Count/ml | Saline Control | Reduction |
| ATHE-2 | 4 hours | 37 × 10⁰ | Initial Inoculum | 3.39 |
| (0.02% Tolnaftate; 1% PEG | | 3 × 10¹ | Population | |
| 400; 0.56% Tween ® 80; | | 0 × 10² | 84, 82 × 10⁴ | |
| 0.46% octoxynol 40) | | 1 × 10³ | 10, 8 × 10⁵ | |
| | | 0 × 10⁴ | 0.1 ml inoculated into | |
| | | 0 × 10⁵ | 10 ml of ATHE-2 | |
| | | | solution | |
| | | | Inoculum population | |
| | | | 8.3 × 10⁴ per ml | |
| | 6 hours | 28 × 10⁰ | Initial Inoculum | 3.46 |
| | | 3 × 10¹ | Population | |
| | | 0 × 10² | 84, 82 × 10⁴ | |
| | | 0 × 10³ | 10, 8 × 10⁵ | |
| | | 0 × 10⁴ | 0.1 ml inoculated into | |
| | | 0 × 10⁵ | 10 ml of ATHE-2 | |
| | | | solution | |
| | | | Inoculum population | |
| | | | 8.3 × 10⁴ per ml | |
| | 24 hours | 9 × 10⁰ | Initial Inoculum | 3.94 |
| | | 1 × 10¹ | Population | |
| | | 0 × 10² | 84, 82 × 10⁴ | |
| | | 0 × 10³ | 10, 8 × 10⁵ | |
| | | 0 × 10⁴ | 0.1 ml inoculated into | |
| | | 0 × 10⁵ | 10 ml of ATHE-2 | |
| | | | solution | |
| | | | Inoculum population | |
| | | | 8.3 × 10⁴ per ml | |
| ATHE-1 | 4 hours | >80 × 10⁰ | Initial Inoculum | 1.29 |
| (0.01% Tolnaftate; 0.5% | | >80 × 10¹ | Population | |
| PEG 400; 0.55% Tween ® | | 36 × 10² | 84, 82 × 10⁴ | |
| 80; 0.46% octoxynol 40) | | 5 × 10³ | 10, 8 × 10⁵ | |
| | | 0 × 10⁴ | 0.1 ml inoculated into | |
| | | 0 × 10⁵ | 10 ml of ATHE-1 | |
| | | | solution | |
| | | | Inoculum population | |
| | | | 8.3 × 10⁴ per ml | |
| | 6 hours | >80 × 10⁰ | Initial Inoculum | 1.30 |
| | | >80 × 10¹ | Population | |
| | | 43 × 10² | 84, 82 × 10⁴ | |
| | | 4 × 10³ | 10, 8 × 10⁵ | |
| | | 0 × 10⁴ | 0.1 ml inoculated into | |
| | | 0 × 10⁵ | 10 ml of ATHE-1 | |
| | | | solution | |
| | | | Inoculum population | |
| | | | 8.3 × 10⁴ per ml | |
| | 24 hours | >80 × 10⁰ | Initial Inoculum | 1.39 |
| | | >80 × 10¹ | Population | |
| | | 28 × 10² | 84, 82 × 10⁴ | |
| | | 4 × 10³ | 10, 8 × 10⁵ | |
| | | 1 × 10⁴ | 0.1 ml inoculated into | |
| | | 0 × 10⁵ | 10 ml of ATHE-1 | |
| | | | solution | |
| | | | Inoculum population | |
| | | | 8.3 × 10⁴ per ml | |
| (ATHE-3) | 4 hours | 0 × 10⁰ | Initial Inoculum | 4.91 |
| (0.02% Miconozole Nitrate; | | 0 × 10¹ | Population | |
| 1% PEG 400; 0.55% (w/w) | | 0 × 10² | 84, 82 × 10⁴ | |
| Tween ® 80; 0.46% | | 0 × 10³ | 10, 8 × 10⁵ | |
| octoxynol 40) | | 0 × 10⁴ | 0.1 ml inoculated into | |
| | | 0 × 10⁵ | 10 ml of ATHE-3 | |
| | | | solution | |
| | | | Inoculum population | |
| | | | 8.3 × 10⁴ per ml | |
| | 6 hours | 0 × 10⁰ | Initial Inoculum | 4.91 |
| | | 0 × 10¹ | Population | |
| | | 0 × 10² | 84, 82 × 10⁴ | |
| | | 0 × 10³ | 10, 8 × 10⁵ | |
| | | 0 × 10⁴ | 0.1 ml inoculated into | |
| | | 0 × 10⁵ | 10 ml of ATHE-3 | |
| | | | solution | |
| | | | Inoculum population | |
| | | | 8.3 × 10⁴ per ml | |
| | 24 hours | 0 × 10⁰ | Initial Inoculum | 4.91 |
| | | 0 × 10¹ | Population | |
| | | 0 × 10² | 84, 82 × 10⁴ | |
| | | 0 × 10³ | 10, 8 × 10⁵ | |
| | | 0 × 10⁴ | 0.1 ml inoculated into | |
| | | 0 × 10⁵ | 10 ml of ATHE-3 | |
| | | | solution | |
| | | | Inoculum population | |
| | | | 8.3 × 10⁴ per ml | |

### EXAMPLE VI

Ocular Irritation tests (Standard Operating Procedure (SOP) 16G-45, Ocular Irritation Test (ISO)) were conducted (Pacific BioLabs, Hercules Calif., USA) for aqueous solutions of the present invention comprising 0.01% (w/w)-0.2% (w/w) miconazole nitrate. The tests were conducted to determine the potential irritating properties of solutions of aqueous solutions comprising the antifungal concentrate of the invention by assessment in an eye irritation test in three white female New Zealand rabbits weighing between 2.5 to 2.9 kg. The aqueous solutions, which were prepared according to the present invention, Lot 061360A (preparation is described in Example II) and Lot 061360B (preparation is described in Example III) were tested. 0.1 mL of the test solution, either Lot 061360A or Lot 061360B, was placed into the right eye of each rabbit. Administration of the test solution was completed by gently pulling each lower eyelid away from the rabbit eyeball to form a cup into which the test solution was dropped. Each eyelid was then gently held together for approximately one second to prevent loss of the test solution. The left eye of each rabbit served as an untreated control. Administration of the test solution, the eyes were examined 1, 24, 48, and 72 hours after treatment. At each observation period, the ocular reaction to treatment was graded according to a numerical scoring system for evidence of corneal ulceration or opacity, inflammation of the iris, or redness, chemosis, and any discharge of the conjunctivae (Biological Evaluation of Medical Devices-Part 10: Tests for Sensitization and Irritation. ISO 10993-10:2002(E)). All grades are based upon on a scale increasing in range from 0-4, depending upon the specific criteria being observed. All animals remained healthy throughout the study period. No irritation in vivo was observed with Lot 061306A (See Table 2) or Lot 061306B (See Table 3) throughout the study period.

TABLE 2
Ocular Irritation Scores for Lot 061360A

| | | | | Cornea | | | | | | Conjunctivae | | | | | |
|---|---|---|---|---|---|---|---|---|---|---|---|---|---|---|---|
| | | | | | | Percent | | | | Red- | | | | | |
| Animal | | Wt. | Time after | Opacity | | Area | | Iris | | ness | | Chemosis | | Discharge | |
| Number | Sex | (kg) | Dosing (hrs) | L | R | L | R | L | R | R | L | L | R | L | R |
| 40833 | F | 2.7 | 1 | 0 | 0 | 0 | 0 | 0 | 0 | 0 | 0 | 0 | 0 | 0 | 0 |
| | | | 24 | 0 | 0 | 0 | 0 | 0 | 0 | 0 | 0 | 0 | 0 | 0 | 0 |
| | | | 48 | 0 | 0 | 0 | 0 | 0 | 0 | 0 | 0 | 0 | 0 | 0 | 0 |
| | | | 72 | 0 | 0 | 0 | 0 | 0 | 0 | 0 | 0 | 0 | 0 | 0 | 0 |
| 40871 | F | 2.6 | 1 | 0 | 0 | 0 | 0 | 0 | 0 | 0 | 0 | 0 | 0 | 0 | 0 |
| | | | 24 | 0 | 0 | 0 | 0 | 0 | 0 | 0 | 0 | 0 | 0 | 0 | 0 |
| | | | 48 | 0 | 0 | 0 | 0 | 0 | 0 | 0 | 0 | 0 | 0 | 0 | 0 |
| | | | 72 | 0 | 0 | 0 | 0 | 0 | 0 | 0 | 0 | 0 | 0 | 0 | 0 |
| 40874 | F | 2.5 | 1 | 0 | 0 | 0 | 0 | 0 | 0 | 0 | 0 | 0 | 0 | 0 | 0 |
| | | | 24 | 0 | 0 | 0 | 0 | 0 | 0 | 0 | 0 | 0 | 0 | 0 | 0 |
| | | | 48 | 0 | 0 | 0 | 0 | 0 | 0 | 0 | 0 | 0 | 0 | 0 | 0 |
| | | | 72 | 0 | 0 | 0 | 0 | 0 | 0 | 0 | 0 | 0 | 0 | 0 | 0 |

R = Right Eye
L = Left Eye

TABLE 3
Ocular Irritation Scores for Lot 061360B

| | | | | Cornea | | | | | | Conjunctivae | | | | | |
|---|---|---|---|---|---|---|---|---|---|---|---|---|---|---|---|
| | | | | | | Percent | | | | Red- | | | | | |
| Animal | | Wt. | Time after | Opacity | | Area | | Iris | | ness | | Chemosis | | Discharge | |
| Number | Sex | (kg) | Dosing (hrs) | L | R | L | R | L | R | R | L | L | R | L | R |
| 40830 | F | 2.7 | 1 | 0 | 0 | 0 | 0 | 0 | 0 | 0 | 0 | 0 | 0 | 0 | 0 |
| | | | 24 | 0 | 0 | 0 | 0 | 0 | 0 | 0 | 0 | 0 | 0 | 0 | 0 |
| | | | 48 | 0 | 0 | 0 | 0 | 0 | 0 | 0 | 0 | 0 | 0 | 0 | 0 |
| | | | 72 | 0 | 0 | 0 | 0 | 0 | 0 | 0 | 0 | 0 | 0 | 0 | 0 |
| 40831 | F | 2.7 | 1 | 0 | 0 | 0 | 0 | 0 | 0 | 0 | 0 | 0 | 0 | 0 | 0 |
| | | | 24 | 0 | 0 | 0 | 0 | 0 | 0 | 0 | 0 | 0 | 0 | 0 | 0 |
| | | | 48 | 0 | 0 | 0 | 0 | 0 | 0 | 0 | 0 | 0 | 0 | 0 | 0 |
| | | | 72 | 0 | 0 | 0 | 0 | 0 | 0 | 0 | 0 | 0 | 0 | 0 | 0 |
| 40832 | F | 2.9 | 1 | 0 | 0 | 0 | 0 | 0 | 0 | 0 | 0 | 0 | 0 | 0 | 0 |
| | | | 24 | 0 | 0 | 0 | 0 | 0 | 0 | 0 | 0 | 0 | 0 | 0 | 0 |
| | | | 48 | 0 | 0 | 0 | 0 | 0 | 0 | 0 | 0 | 0 | 0 | 0 | 0 |
| | | | 72 | 0 | 0 | 0 | 0 | 0 | 0 | 0 | 0 | 0 | 0 | 0 | 0 |

R = Right Eye
L = Left Eye

### EXAMPLE VII

Cytotoxity studies were also conducted for aqueous solutions comprising the antifungal concentrate of the invention. The cytotoxity test was conducted (Pacific BioLabs, Hercules Calif., USA; SOP 15A-02 and SOP 15B-01) using test materials, Lot 061360A (preparation is described in Example II) and Lot 061360B (preparation is described in Example III), in the United States Pharmacopoeia (USP) standard minimum essential media (MEM) elution test against L-929 mouse fibroblast cells (ATCC Cell Line CCL1, NCTC Clone 929). Test article extract was prepared using 3.0 grams of test material consisting of a representative portion of the test article that was extracted in 15.0 mL of 5% serum supplemented MEM at 37° C.±1 ° C. in a humidified incubator with 5%±1% CO₂ for not less than 24 hours. Positive and negative controls were also extracted under the same conditions. After the 24 hour extraction period, the growth medium from duplicate 10 cm² wells each containing the monolayer of L-929 cells were decanted and replaced with 2 mL of test article extract. The control extracts were tested in the same manner as the extracting medium containing test article. All cell cultures were incubated for 48±2 hours at 37° C.±1° C. in a humidified incubator with 5%±1% CO₂. The cells were scored for cytotoxic response at 48 hours. The Lot 061360A and Lot 061360B aqueous solutions (test materials) each scored a 0 (zero) indicating no biological reactivity (i.e. no cytotoxicity). Possible biological reactivity scores were 0 (zero) indicating no reactivity, 1 indicating Slight Reactivity, 2 indicating Mild Reactivity, 3 indicating Moderate Reactivity, and 4 indicating Severe Reactivity. According to USP specifications, a test material meets the requirements of the test if the cell culture treated with the test material does not score greater than a grade of 2 (Mild Reactivity). The test results are presented in Table 4 for Lot 061360A and Lot 061360B aqueous solutions (test materials) prepared according to the present invention.

TABLE 4
Cytotoxicity Scores

| Test Material | Grade | Biological Reactivity |
|---|---|---|
| 61360A | 0 | None |
| 061360B | 0 | None |
| Positive Control Extract | 4 | Severe |
| Negative Control Extract | 0 | None |

We claim:

1. A method for preparing a concentrate solution containing an antifungal agent in a form suitable for subsequent introduction in an aqueous medium suitable for ophthalmic or periophthalmic use, the method comprising the steps of:
a) dissolving an antifungal agent in a physiologically acceptable non-aqueous vehicle to form a non-aqueous solution of said antifungal agent said vehicle being miscible with aqueous solutions;
b) adding a physiologically acceptable surfactant to said non-aqueous solution in an amount sufficient to stabilize said antifungal agent upon its subsequent introduction in said aqueous medium; and
c) mixing the non-aqueous solution and surfactant mixture until a clear concentrate solution is obtained.

2. The method of claim 1, wherein the antifungal agent is added in a concentration within the range of 0.005% (w/w) to 6.0% (w/w).

3. The method of claim 1, wherein the surfactant is added in a concentration within the range from 0.5% (w/w) to 2.0% (w/w).

4. The method of claim 1, wherein said vehicle is polyethylene glycol having a molecular weight within the range from 8 to 1000.

5. The method of claim 1, wherein said surfactant is polysorbate 80.

6. The method of claim 1, wherein said antifungal agent is selected from the group consisting of amorolfine, amphotericin B, anidulafungin, butoconazole, butenafine, caspofungin, ciclopirox olamine, clotrimazole, econazole, fluconazole, flucytosine, griseofulvin, haloprogin, itraconazole, ketoconazole, micafungin, miconazole, miconazole nitrate, naftifine, nikkomycin Z, topical nystatin, liposomal nystatin, oxiconazole, posaconazole, pimaricin, ravuconazole, sulconazole, terbinafine, terconazole, tioconazole, tolnaftate, undecylenate, and voriconazole.

7. A method for inhibiting fungal growth in an aqueous solution suitable for ophthalmic or periophthalmic use comprising the step of dissolving in said aqueous solution a concentrate solution containing an antifungal agent dissolved in a physiologically acceptable non-aqueous vehicle, said vehicle being miscible with said aqueous solution and one or more physiologically acceptable surfactants, said concentrate solution being added to said aqueous solution in an amount sufficient to provide a fungicidally or fungistatically effective amount of said antifungal agent in said aqueous solution.

8. The method of claim 7, wherein said vehicle is polyethylene glycol having a molecular weight within the range from 8 to 1000.

9. The method of claim 7, wherein said antifungal agent is selected from the group consisting of amorolfine, amphotericin B, anidulafungin, butoconazole, butenafine, caspofungin, ciclopirox olamine, clotrimazole, econazole, fluconazole, flucytosine, griseofulvin, haloprogin, itraconazole, ketoconazole, micafungin, miconazole, miconazole nitrate, naftifine, nikkomycin Z, topical nystatin, liposomal nystatin, oxiconazole, posaconazole, pimaricin, ravuconazole, sulconazole, terbinafine, terconazole, tioconazole, tolnaftate, undecylenate, and voriconazole.

10. A concentrate solution containing an antifungal agent in a form suitable for subsequent introduction in an aqueous medium suitable for ophthalmic or periophthalmic use, said concentrate solution made by:
a) dissolving an antifungal agent in a physiologically acceptable non-aqueous vehicle to form a non-aqueous solution of said antifungal agent said vehicle being miscible with aqueous solutions;
b) adding a physiologically acceptable surfactant to said non-aqueous solution in an amount sufficient to stabilize said antifungal agent upon its subsequent introduction in said aqueous medium; and
c) mixing the non-aqueous solution and surfactant mixture until a clear concentrate solution is obtained.

11. The concentrate of claim 10, wherein the antifungal agent is added in a concentration within the range of 0.005% (w/w) to 6.0% (w/w).

12. The concentrate of claim 10, wherein the surfactant is added in a concentration within the range from 0.5% (w/w) to 2.0 (w/w).

13. The concentrate of claim 10, wherein said vehicle is polyethylene glycol having a molecular weight within the range from 8 to 1000.

14. The concentrate of claim 10, wherein said surfactant is polysorbate 80.

15. The concentrate of claim 10, wherein said antifungal agent is selected from the group consisting of amorolfine, amphotericin B, anidulafungin, butoconazole, butenafine, caspofungin, ciclopirox olamine, clotrimazole, econazole, fluconazole, flucytosine, griseofulvin, haloprogin, itraconazole, ketoconazole, micafungin, miconazole, miconazole nitrate, naftifine, nikkomycin Z, topical nystatin, liposomal nystatin, oxiconazole, posaconazole, pimaricin, ravuconazole, sulconazole, terbinafine, terconazole, tioconazole, tolnaftate, undecylenate, and voriconazole.

16. An aqueous solution for ophthalmic or periophthalmic use comprising an antifungal agent in an amount effective to inhibit fungal growth in said aqueous solution, said solution made by dissolving in first aqueous solution containing no antifungal agent a second concentrate solution containing (a) an antifungal agent dissolved in a physiologically acceptable non-aqueous vehicle, said vehicle being miscible with said first aqueous solution containing no antifungal agent; and (b) one or more physiologically acceptable surfactants; said concentrate having been added to the first aqueous solution containing no antifungal agent in an amount sufficient to provide a fungicidally or fungistatically effective amount of said antifungal agent in said first aqueous solution.

17. The method according to claims 1 or 7, wherein the antifungal agent is tolnaftate.

18. The method according to claims 1 or 7, wherein the antifungal agent is miconazole nitrate.

19. The method according to claims 1 or 7, wherein said vehicle or solvent is polyethylene glycol 400.

20. The method according to claims 1 or 7, wherein said surfactants are polysorbate 80 and octoxynol 40.

21. The method according to claims 1 or 7, wherein said aqueous solution is a multipurpose lens solution.

22. The aqueous solution of claim 16, wherein the antifungal agent is selected from the group consisting of amorolfine, amphotericin B, anidulafungin, butoconazole, butenafine, caspofungin, ciclopirox olamine, clotrimazole, econazole, fluconazole, flucytosine, griseofulvin, haloprogin, itraconazole, ketoconazole, micafungin, miconazole, miconazole nitrate, naftifine, nikkomycin Z, topical nystatin, liposomal nystatin, oxiconazole, posaconazole, pimaricin, ravuconazole, sulconazole, terbinafine, terconazole, tioconazole, tolnaftate, undecylenate, and voriconazole.

23. A concentrate solution containing 1.0% (w/w) miconazole nitrate in a form suitable for subsequent introduction in an aqueous medium suitable for ophthalmic or periophthalmic use, said concentrate solution made by:
a) dissolving 1.0% (w/w) miconazole nitrate in 50% (w/w) polyethylene glycol with continual mixing at a temperature of 75° C.-85° C. to form a non-aqueous solution of said antifungal agent upon its subsequent introduction in said aqueous medium, said vehicle being miscible with aqueous solutions;
b) cooling said non-aqueous solution to 30° C.-40° C.;
c) adding 27.5% (w/w) polysorbate 80 and 22.1% (w/w) octoxynol 40 to said non aqueous solution in an amount sufficient to stabilize said miconzole nitrate upon its subsequent introduction in said aqueous medium; and
d) mixing the non-aqueous solution and polysorbate 80 and octoxynol 40 mixture until a clear concentrate solution is obtained.

24. A concentrate solution for ophthalmic or periophthalmic use containing from about 0.005% (w/w) to about 6.0 % (w/w) antifungal agent, from about 24% (w/w) to about 79% (w/w) non-aqueous vehicle, and from about 20% (w/w) to about 70.0% (w/w) surfactant.

25. A concentrate solution for ophthalmic or periophthalmic use containing from about 0.005% (w/w) to about 6.0 % (w/w) antifungal agent tolnaftate or miconazole nitrate, from about 24% (w/w) to about 79% (w/w) non-aqueous vehicle polyethylene glycol 400, and from about 20% (w/w) to about 70.0% (w/w) surfactants polysorbate 80 and octoxynol 40.

26. The concentrate solution of claim 24 or claim 25, wherein said concentrate solution is dissolved in a first aqueous solution to produce a second aqueous solution, wherein said second aqueous solution is for ophthalmic or periophthalmic use.

27. An aqueous solution for ophthalmic or periophthalmic use comprising from about 0.001% (w/w) to about 2.0% (w/w) miconazole nitrate, from about 0.5 (w/w) to about 1.6% (w/w) polyethylene glycol 400, from about 0.3% (w/w) to about 10.0% (w/w) polysorbate 80 and from about 0.3% (w/w) to about 10.0% (w/w) octoxynol 40, and from about 0.0001% (w/w) to about 0.0002% (w/w) polyhexamethylene biguanide.

28. An aqueous solution for ophthalmic or periophthalmic use comprising from about 0.001% (w/w) to about 2.0% (w/w) miconazole nitrate, from about 0.5% (w/w) to about 1.6% (w/w) polyethylene glycol 400, from about 0.3% (w/w) to about 10.0% (w/w) polysorbate 80 and from about 0.3% (w/w) to about 10.0% (w/w) octoxynol 40, and from about 0.001% (w/w) to about 0.002% (w/w) polyquaternium-1.

29. An aqueous solution for ophthalmic or periophthalmic use comprising from about 0.001% (w/w) to about 2.0% (w/w) tolnftate, from about 0.5% (w/w) to about 1.6% (w/w) polyethylene glycol 400, from about 0.3% (w/w) to about 10.0% (w/w) polysorbate 80 and from about 0.3% (w/w) to about 10.0% (w/w) octoxynol 40.

30. A method for avoiding fungal infection in a subject, said infection arising from contact of the eye of said subject with an aqueous solution during ophthalmic or periophthalmic use of said aqueous solution, the method comprising: providing as the aqueous solution used ophthalmically or periophthalmically an aqueous solution suitable for ophthalmic or periophthalmic use to which has been added a concentrate comprising (a) an antifungal agent dissolved in a non-aqueous vehicle, said vehicle being miscible with an aqueous solution suitable for ophthalmic or periophthalmic use containing no antifungal agent, and (b) one or more physiologically acceptable surfactants, said concentrate having been added in an amount sufficient to provide a fungicidally or fungistatically effective amount of said antifungal agent in said provided aqueous solution.
